(19) United States
(12) Patent Application Publication
(10) Pub. No.: US 2005/0027216 A1
(43) Pub. Date: Feb. 3, 2005

(54) Portable detector for measuring movements of a person wearing the detector, and method

(75) Inventors: Regis Guillemaud, La Tronche (FR); Yanis Caritu, St. Joseph De Riviere (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 10/879,303

(22) Filed: Jun. 30, 2004

(30) Foreign Application Priority Data
Jul. 2, 2003 (FR) 03 50285

(51) Int. Cl. A61B 5/11

(52) U.S. Cl. 600/595

(57) ABSTRACT

This detection device comprises processing means (6) for distinguishing the movements of a person wearing a detector due to an external activity from movements due to his physiological activity. Movement sensor signals (4, 5) are filtered in different ways. The external activity is estimated and a subtraction gives the results for the physiological activity. Special processing is done to take account of exceptional states of the activity, such as sudden movement variations. Results can also be improves by discerning the type of activity being performed by the wearer. Finally, it is advantageous if several different sensors measure movements in different directions and if the most important measurements are chosen.

The subject of this invention is a portable detector designed to measure the movements of a person wearing the detector, and a corresponding method.

There is a very wide variety of prior art for measuring heart or other signals using a detector that is attached to a patient. Movement sensors such as accelerometers have thus been proposed to monitor movements of the thorax cage and to deduce the heart rate from these movements. However, this type of detector has been reserved for particular conditions or postures of the patient; in general, a lack of effort or movement is necessary to give a reliable measurement, that is not confused by components from sources other than the movement signal, and that could be preponderant due to the small amplitude of movements originating from the heart.

Movement sensors at various locations of the body have also been applied to monitor persons wearing the detector and sometimes to determine a state of sleep, a fall, etc. The complexity of postures and levels of human activity makes a real analysis of the activity difficult when using usual detectors, which are reserved particularly for the detection of a single type of event and are programmed to ignore other events, as far as possible.

For example, it would be useful to complete a fall detector with a physiological measurements detector to check the state of the patient after the fall, but this would be only possible if the patient wears two corresponding detectors, which is uncomfortable.

The invention proposes an improved portable detector, characterized by signal processing means for distinguishing a signal component due to an external activity of the wearer, at least one signal component due to a physiological activity (heart beats or breath in particular). Thus, as we have seen, known detectors are designed either to measure the physiological activity or the external activity. They are almost reduced to a sensor or group of sensors collecting the movement signal or signals, and means of reading and transmitting the signals that are interpreted directly.

The purposes of the invention are to:
supply a detector making a distinction between signal components with clearly different levels and that can vary strongly with time;
doing the above, starting from measurements made by the same movement sensors;
provide such a detector with a unit structure and that is compact;
offer an increased capacity for measurement and diagnosis of physiological states, limiting the durations in which measurements must not be considered;
offer a more universal determination of posture and activity states of the wearer, and discern a larger number of them.

Heart beats and breathing are periodic movements, for which the intensity and frequency vary depending on the activity level of the wearer under particular conditions. Movements due to the external activity of the wearer are usually low frequency; but since they are not periodic, they cover a wider frequency range, and their intensity can vary strongly. it is impossible to separate these movements by simple signal filtering, due to these frequency variations and even more to overlaps of frequency bands associated with these different movements. However, satisfactory results have been obtained by applying a non-stationary filter to the movement signal or signals by subtracting the filtered signal from the original signal (the unprocessed signal or a signal on which preliminary filtering has been done to eliminate noise); the original physiological signals are then quite well defined.

Incorrect detection that can lead to a false alert should be avoided. This type of situation can arise with some particularly sudden external movements that actually prevent satisfactory detection of physiological movements. Therefore, it would be useful to add a module to the detector to recognize such a situation according to criteria that depend on the external activity component, and which is used to temporarily invalidate the estimate of the component due to the physiological activity.

Another difficulty is the sensitivity of measurements to the body posture adopted by the wearer, since the acceleration due to gravity which is involved in accelerometric measurements and that has to be corrected, is perceived with an intensity that depends on this posture, and since measurements of the physiological activity give much lower acceleration values. It is recommended that wearer position indicators should be added, particularly magnetometers measuring the direction of the ambient magnetic field in order to clearly determine the posture of the wearer and to choose only some of the movement signals, while eliminating signals that are excessively affected by gravity, for treatment according to the invention. This improvement is useful particularly when several sensors measure different wearer movements in different directions. One frequent situation consists of using three sensors, measuring movements in perpendicular directions, usually one forward movement, one sideways movement and one upward movement of the person.

One embodiment of the invention will now be described more completely with reference to the figures.

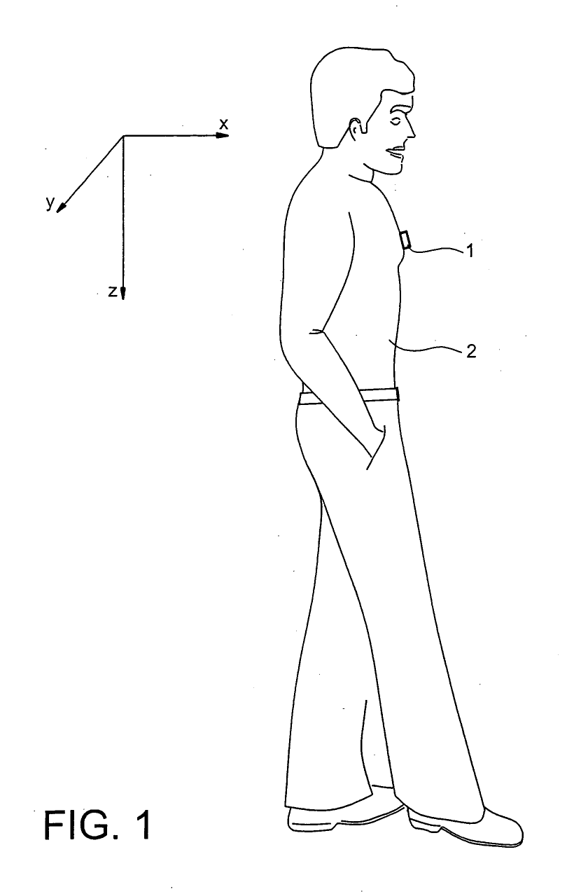

FIG. 1 illustrates the position of the detector on the wearer,

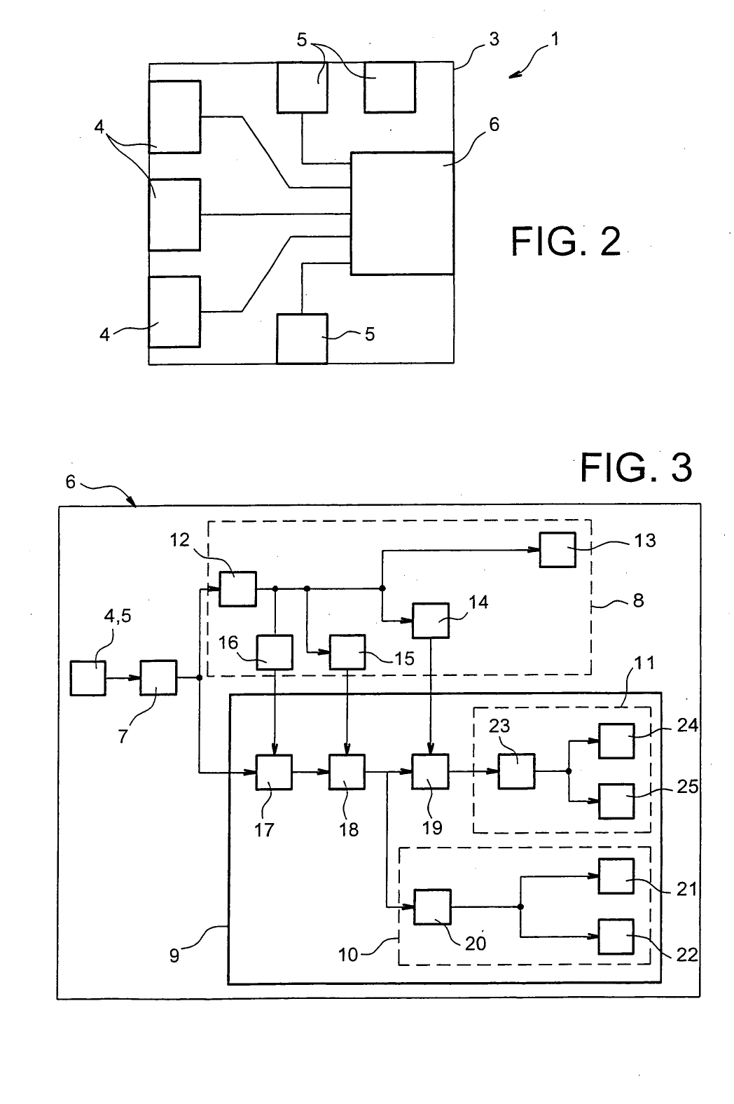

FIG. 2 shows the detector as a whole, and

FIG. 3 shows the processing system.

FIG. 1 shows that the detector marked as reference 1 is placed on the chest of the wearer 2. It would be placed on the abdomen or elsewhere. The detector 1 is miniature so that, unlike others, it can be worn comfortably almost unperceived. The X, Y and Z axes are introduced to facilitate the explanation and define a coordinate system related to the wearer 2, the X axis being in the forward direction, the Z axis being downwards towards the wearer's feet, and the Y axis is being towards the right.

According to FIG. 2, the detector 1 may comprise a unit 3 containing three accelerometers all marked as reference 4, three magnetometers all marked as reference 5, and a processing system 6 to which the accelerometers 4 and magnetometers 5 are connected by wires through which their signals are carried to it. Each accelerometer 4 measures an acceleration component of the chest movement of the wearer 2 along one of the X, Y and Z axes, as a function of the direction of gravity; the magnetometers 5 do the same thing as a function of the direction of the earth's magnetic field. The detector 1 is kept at a constant orientation in contact with the skin or clothing of the wearer 2 by glue, a seam, a clamping strip or any other suitable means. The processing unit 6 will now be described with reference to FIG. 3.

The signals output from the accelerometers 4 or magnetometers 5 each pass through a normalization module 7 and are transmitted to two calculation modules 8 and 9 working in parallel and in interaction, the first (8) of which calculates the component of the signals due to external activity of the wearer 2, and the second (9) of which calculates the component of the signals due to the physiological activity; this second module 9 comprises a sub-module 10 assigned to movements due to heart beats and a sub-module 11 assigned to movements due to breathing.

The first calculation module 8 comprises a low pass filter 12 that transmits the signal output from the normalization module 7 to an activity analysis device 13, to a posture analysis device 14, an activity level analysis device 15 and a device 16 for estimating the activity component. The signal output from the normalization module 7 reaches sub-modules 10 and 11 after passing through a subtractor 17, a validation module 18 and also a selection device 19 for the sub-module 11. The sub-module 10 comprises a device for extraction of the heart component 20, a frequency calculation device 21 and an examination device. 22. The sub-module 11 comprises a device for extraction of the breathing component 23, a device for the frequency calculation 24 and an output device 25.

These various elements will be described in sequence and in detail. The normalization device 7 is of an ordinary type that is used to calibrate the signals, for example according to a linear law, to supply normalized output signals that are proportional to the acceleration applied to them. The low pass filter 12 is used to eliminate signal high frequencies that in practice only express noise. The activity analysis device 13 is not indispensable and its content may depend on the activity types to be diagnosed, such as a fall, sleep, walking, position change or others. The diagnosis can be made-with several sensors 4 and 5. The posture analysis device 14 can determine if the wearer 2 is standing up, seated or lying down, by comparing accelerations measured by accelerometers 4. If the largest signal is measured by the accelerometer 4 along X or the accelerator 4 along Y, the wearer is lying down, but the acceleration along Z will be preponderant if he is seated or standing, since gravity acts along this axis. The posture diagnosis is made if the acceleration ratios are higher than some specific coefficients. If the wearer 2 is standing up, the comparison of measurements for magnetometers 5 along X and Y can give its direction along the cardinal points. A fall can be determined if a fast rotation is detected about a vertical axis or a fast acceleration in rotation with respect to the field of gravity (measured with an accelerometer). Other criteria can easily be deduced for other postures.

The activity level analysis device 15 is designed to indicate if the activity of the wearer 2 reaches a level beyond which it is considered to be impossible to obtain the results for the physiological measurements correctly. It may consist of a bypass filter applied to signals from sensors 4 and 5 and produces a binary output. If the derived signal is more than a threshold, which is the result of an excessively sudden movement variation, the device 15 supplies an output equal to zero, and otherwise the output is equal to one. Another way of proceeding would be to apply a sliding criterion on differentiated signals originating from the sensors, according to the following formula:
CRI=Abs [d(t)−d(t−k)]Sign[d(t).d(t−k)]
where CRI is the criterion, Abs is the absolute value operator, d is the differentiated signal originating from a sensor, t is the time, k is a predefined constant and Sign is the sign operator; the device 15 will have a zero output if the calculation result is less than a negative threshold, which corresponds to a fast inversion of the movement direction, and otherwise the output will be equal to one.

When the signal from device 15 is zero, the validation module 18, which is a multiplier, outputs a null signal and therefore inhibits calculations of the physiological activity; otherwise, when the device 15 outputs a signal equal to 1, the validation module 18 has no influence over the signal passing through it and allows it to pass through without modifying it.

The purpose of the estimating device 16 is to isolate a component of the signal from each sensor 4 or 5 representative of the wearer's activity. It may be a filter like a low pass filter, or more usefully a non-stationary filter used to avoid filtering the signal in the presence of a singular point of the signal corresponding to a fast inversion of its movement.

A filter F using a sigmoid function may be used. This process is based on the concept that the signal may be filtered without any disadvantage when it is stable, but it must not be filtered in highly unstable situations in which the wearer's activity also includes higher frequency movements.

A sigmoid function tends towards 0 for input values close to 0, and towards 1 for very high input values. One example is 1/(1+e^{-x}), but asin, atan and others functions can also be used.

According to the above, a filter on the input signal denoted s(t) may be a low pass filter weighted by the criterion CRI mentioned above:
F[S(t)]=sigmoid (CRI).s(t)+(1−sigmoid (CRI))×F[S(t) low pass].

where the sigmoid is 1/(1+e^{-x})

Filter functions other than F may also be applied, or filters capable of extracting a low frequency component of the signal that maintains discontinuities may also be applied. Another recommended example of a filter is that mentioned in the article “Non linear anisotropic filtering of MRI data” IEEE Transactions on Medical Imaging, vol. 11, No. 2, p. 231-232 by G. Gerig.

The subtractor 17 has a positive terminal into which the normalized signal is input, and a negative terminal into which the signal output by the estimating device 16 is input. The difference corresponds to the signal representing the physiological activity. As we have seen, the validation module 18 is a multiplier that leaves this signal unchanged under circumstances considered to be normal, and otherwise cancels it. The selection device 19 is used to choose the signals that are the most representative of the breathing movement as a function of the posture of the wearer 2 estimated by the posture analysis device 14. If the wearer 2 is lying down, the movements due to breathing will be estimated by accelerometers 4 sensitive along the Y and Z directions, and by magnetometers 5 along the X and Z directions; otherwise, when the wearer 2 is seating or standing, accelerometers 4 will be considered along the X and Z directions and magnetometers 5 will be considered along the Y and Z directions. This provides a means of eliminating accelerometers influenced by the acceleration due to gravity that would supply excessively noisy measurements.

The heart rate extractor 20 is a low pass filter for which the limits may for example be 0.5 Hertz and 3 Hertz. The heart frequency calculation device 21 advantageously uses accelerometers 4 and particularly the accelerometer oriented along the X direction. The period is calculated by detecting consecutive maximums and estimating the durations that separate them. These maximums are produced by the main heart beat; they are about 30 milliseconds wide and are separated on average by a period of about 0.8 seconds for a person at rest. Detection may be improved by applying filtering adapted to the shape of the maximums to be detected, for example a filter with an equivalent width of 250 milliseconds which is a value equal to 1 at the center on an equivalent width of 30 milliseconds, and 0 at the periphery. The heart rate is equal to the inverse of the duration separating the maximums. A sliding average calculation can be made using the average of a few previously measured frequencies into consideration.

The output device 22 is usually a transmitter directing the results obtained towards a display or diagnosis device external to the detector 1.

The breathing component extraction device 23 also comprises a low pass filter between frequencies for example equal to 0.03 Hertz and 1 Hertz. The breathing rate calculation device 24 uses the results from one or several sensors 4 and 5 and calculates the breathing rate by estimating the duration between three consecutive passages of a breathing signal through zero; the rate is the inverse of this duration. In this case, a sliding average calculation can be carried out to improve the results, or an average of the calculation can be made on several sensors 4 and 5. Finally, the output device 25 still transmits results obtained towards an external display or diagnosis means, or a means of synchronizing another instrument on the breathing cycle.

There is no need to place six movement sensors in the detector 1 to use the invention, but it is quite obvious that the measurement of movements in all directions by two series of sensors with different references would give more universal results.

These magnetometers could be differential probes (fluxgates) or giant magneto-resistances.

In another embodiment, the detector comprises several sensors, for example distributed at different locations of the body, each sensor being connected to the signal processing unit 6, for example by an electrical connection, by radiofrequency. The advantage of this embodiment is that it overcomes the inability of a sensor to give physiological information, for example if the patient is leaning on a sensor, so that the sensor can no longer measure breathing. The other sensors located elsewhere are used. The number of sensors used, their degree of redundancy and their locations are not critical.

1) Portable detector (1) comprising at least one sensor (4, 5) collecting a movement signal from a person (2) wearing the detector, characterized in that it comprises signal processing means (6) for distinguishing a component of the signal due to an external activity of the wearer and at least one component of the signal due to a physiological activity of the wearer.

2) Detector according to claim 1, characterized in that it comprises a filter receiving the movement signal, to estimate the component due to external activity, and a subtracting module (17), receiving the movement signal at a positive terminal and the signal output from the filter (16) at a negative terminal to estimate the component due to the physiological activity.

3) Detector according to either claim 1 or 2, characterized in that it comprises an invalidation decision module (15) for estimating the component due to the physiological activity based on a criterion that depends on the external activity component.

4) Detector according to any one of claims 1 to 3, characterized in that it comprises three sensors (4 or 5) collecting three perpendicular movement signals of the person, including one forward movement, one sideways movement and one upward movement.

5) Detector according to any one of claims 1 to 4, characterized in that it comprises several sensors collecting corresponding movement signals including magnetometers, a wearer posture estimating module (14), and a sensor selection means to select some movement signals, that will be applied to processing means, and discard other movement signals, as a function of the wearer's posture.

6) Detector according to claim 2, characterized in that the filter is non-stationary.

7) Fall sensor characterized in that it comprises a detector according to any one of claims 1 to 6.

8) Process for detecting the activity of a person, characterized in that it consists of measuring the movements of the person, and separating movements due to external activity from movements due to a physiological activity.

9) Process according to claim 8, characterized in that it includes filtering of a signal measured to estimate the movements due to the external activity and subtraction of the measured signal for the signal resulting from filtering to estimate the movements due to physiological activity.
